(19) United States
(12) Patent Application Publication
(10) Pub. No.: US 2005/0129708 A1
(43) Pub. Date: Jun. 16, 2005

(54) Fucoidan-based health food

(76) Inventors: Makoto Fujii, Kagoshima-shi (JP); De-Xing Hou, Kagoshima-shi (JP); Masaji Nakamizo, Fukuoka-shi (JP)

Correspondence Address: JORDAN AND HAMBURG LLP, 122 EAST 42ND STREET, SUITE 4000, NEW YORK, NY, 10168, US

(21) Appl. No.: 10/735,958

(22) Filed: Dec. 15, 2003

(51) Int. Cl. A61K 35/84; A61K 31/715

(52) U.S. Cl. 424/195.15; 514/54

(57) ABSTRACT

Purpose: To find out conditions for using a fucoidan-based polysaccharide composite extracted from sprouts of wakame as a mixture in order to extensively utilize the excellent function as bioprotective accelerator and immunostimulator possessed thereby for maintenance of health in human life. Solution: A fucoidan-based health food having the effect of inhibiting hyperglycemia and keeping blood sugar low is obtained by mixing an Agaricus powder to a mixture consisting of 1-90% of a fucoidan fraction extraction-fractionated from sprouts of wakame and the remainder of a fucoidan fraction extraction-fractionated from Okinawa Tinocladia crassa. A preferable formula composition thereof comprises the Agaricus powder mixed to a mixture of consisting of 15-35% by mass of a fucoidan fraction extraction-fractionated from sprouts of wakame and 85-65% by mass of a fucoidan fraction extraction-fractionated from Okinawa Tinocladia crassa in a content of 10-20% by mass based on the whole amount of the mixture.

### DETAILED DESCRIPTION OF THE INVENTION

1. Field of the Invention

The present invention relates to a fucoidan-based health food that is orally administered as a powder, a capsule, a tablet or a drink dissolved in a solvent such as water or a seasoned solution to make it easy to drink.

2. Description of the Related Art

Conventionally, health foods for reducing blood sugar level to bring about a dietary effect are variously known. Particularly, fucoidan-like polysaccharide composites are known to have the function of improving bioprotective ability potentiating activities by enhancing the activities of the natural killer cells (NK) that prevent canceration of an organism by removal of foreign matters and enhancing the sthenic action on phagocytosis of macrophage (M φ) that is one of the important members to establish immunity. For example, a method for providing such a polysaccharide at a low cost is disclosed in Japanese Patent Application Laid-Open No. 2001-181303, which comprises cold-drying sprouts of wakame (Undaria pinnatifida) followed by low-temperature drying and pulverizing thereof.

3. Problem to be Solved by the Invention

The fucoidan-like polysaccharide composite extracted from sprouts of wakame itself has an excellent function as bioprotective accelerator and immunostimulator. However, when mixed with another health food material, the function might be deteriorated, or competitively deteriorated with additive components added thereto.

Such a fucoidan-like polysaccharide composite further has the defect of lacking the taste and flavor as health food because of the excessively strong distinctive flavor of the sprouts of wakame that are the raw materials.

In order to extensively use the excellent function as bioprotective accelerator and immunostimulator possessed by the fucoidan-LIKE polysaccharide composite extracted from sprouts of wakame for maintenance of health in human life, the first subject of the present invention is to find out a condition for using it as a mixture.

The other subject of the present invention is to find out an additive which never deteriorates the function as bioprotective accelerator and immunostimulator possessed by the fucoidan-like polysaccharide composite extracted from sprouts of wakame.

Another subject of the present invention is to find out an additive capable of enhancing the function possessed by the fucoidan-like polysaccharide composite extracted from sprouts of wakame or exhibiting a new function without deteriorating the function possessed by the fucoidan-like polysaccharide composite itself.

The other subject of the present invention is to confirm the minimum content enable the survival of the function of the fucoidan-like polysaccharide composition extracted from sprouts of wakame, when mixed with an additive compound.

Further, another subject of the present invention is to confirm the upper limit value of the content enabling the exhibition of the function of the fucoidan-like polysaccharide composition extracted from sprouts of wakame, when mixed with another additive.

The other subject of the present invention is to confirm a condition for laying the fucoidan-like polysaccharide composite extracted from sprouts of wakame in a stat easy to drink.

4. Means to Solve the Subjects

The present invention has been accomplished under the knowledge that the function of the fucoidan extraction-fractionated from sprouts of wakame is not deteriorated but adversely enhanced by being mixed with a specified quantity of a fucoidan extraction-fractionated from Okinawa Tinocladia crassa, and the function expected as health food is further additively enhanced with excellent easiness to drink by adding an Agaricus powder containing β-glucan that is a Brazilian mushroom.

Namely, by mixing a specified quantity of the fucoidan extraction-fractionated from Okinawa Tinocladia crassa, a fucoidan-based health food additionally having the function of the fucoidan extraction-fractionated from Okinawa Tinocladia crassa without deteriorating the function of the fucoidan extraction-fractionated from sprouts of wakame, and improved in easiness to drink can be obtained.

As the additive compound for enhancing the function possessed by the fucoidan extraction-fractionated from sprouts of wakame, powder of a mushroom of the genus Agaricus (Brazilian mushroom) containing β-glucan or powder of a mushroom such as Agaricus brazei Murill (himematutake), which is said to give anticancer function to the function of the fucoidan extraction-fractionated from sprouts of wakame is suitably usable and excellent in flavor.

By mixing the Okinawa Tinocladia crassa-derived fucoidan that is expected to have the same function and the Agaricus that is expected to enhance the immunity in specified ratios, respectively, the function of the wakame sprout-derived fucoidan of effectively controlling body weight and reducing fat tissue weight, neutral fat (triglyceride) in serum, low-density lipoprotein and total cholesterol content can be exhibited synergistically with the respective functions without being inhibited at all to enhance the activities of the natural killer cells (NK) of preventing the canceration of an organism through removal of mutant cells (abnormal or cancerated cells) or provide bioprotective ability potentiating activity.

Namely, the fucoidan-based health food of the present invention has the effect of inhibiting hyperglycemia and keeping blood sugar low by mixing an Agaricus powder to a mixture consisting of 1-90% of a fucoidan fraction extraction-fractionated from sprouts of wakame and the remainder of a fucoidan fraction extraction-fractionated from Okinawa Tinocladia crassa.

A preferred formula composition of the fucoidan-based health food comprises the Agaricus powder mixed to a mixture consisting of 15-35% by mass of the fucoidan fraction extraction-fractionated from sprouts of wakame and 85-65% by mass of the fucoidan fraction extraction-fractionated from Okinawa Tinocladia crassa in a content of 10-20% by mass based on the whole amount of the mixture.

It is 1% or more that the fucoidan (sulfated polysaccharide) fraction extraction-fractionated from sprouts of wakame can exhibit its function, and when it is 90% or more, the flavor of the wakame sprouts that are the raw materials is too strong.

As the compounding agent to be added to the fucoidan extraction-fractionated from the wakame sprouts, fucoidans extracted from general Nemacystis decipiens can be used. Among them, the fucoidan extracted from Okinawa Tinocladia crassa is most suitable as the compounding agent to the fucoidan extraction-fractionated from wakame sprouts because it exhibits the similar function although the function level is low, compared with the fucoidan extracted from sprouts of wakame, and is more excellent in flavor than the fucoidan prepared from wakame sprouts.

The mixture of wakame sprouts and Okinawa Tinocladia crassa can be replaced by extracts from other foreign seaweeds to the extent that its characteristics are never lost.

Even if the mixed Agaricus is replaced by Agaricus brazei Murill (himematutake) that is said to have the same function as the Agaricus in a small quantity (up to about 10%), the functions and flavor of the health food of the present invention are never lost. Accordingly, the Agaricus referred to in the present invention collectively means those partially substituted by such an alternative.

In the fucoidan-based health food of the present invention, thus, the functions of the Okinawa Tinocladia crassa and the Agaricus powder can be added to and exhibited synergistically with the function of the wakame sprout-derived fucoidan (sulfated polysaccharide) having the body weight control effect and the reducing effect of fat tissue weight, neutral fat in serum (triglyceride), low-density lipoprotein and total cholesterol content as the base material without inhibiting its function, and the easiness to drink of the wakame sprout-derived fucoidan can be also improved.

Since the fucoidan-based health food of the present invention, which is powdery by nature and served as a powder or tablet, is essentially water-soluble, it can be served as a general drink by dissolving it in water or a seasoned solution, or as a mixture with another food material.

### BRIEF DESCRIPTION OF THE DRAWINGS

FIG. 1 shows the body weight change of rats fed for 1 month;

FIG. 2 shows the neutral fat content in serum of rats fed for one month;

FIG. 3 shows the change in total cholesterol content in serum of rats fed for one month;

FIG. 4 shows the change in low-density (LDL) cholesterol and high-density (HDL) cholesterol contents of rats fed for one month;

FIG. 5 shows the change in fat tissue weight of rats fed for one month;

FIG. 6 shows the change in natural killer activity in spleen of mice fed for 20 days;

FIG. 7 shows the change in tumor weight caused by Sarcoma 180 of mice fed on the fucoidan-based health food of the present invention for 20 days after transplanting Sarcoma 180;

FIG. 8 shows the change in natural killer activity in spleen of mice fed on the fucoidan-based health food of the present invention for 20 days after transplanting Sarcoma 180; and

FIG. 9 shows the change in blood sugar value of model mice of diabetes KK-Ay fed for 70 days.

### PREFERRED EMBODIMENTS OF THE INVENTION

The present invention will be further described in detail according to a preferred embodiment.

1. Preparation of Compounding Raw Materials Fucoidan Fraction Extraction-Fractionated from Sprouts of Wakame

Sprouts of wakame were collected and washed with seawater followed by sun drying all day and night, and then cold-dried. The water content was 6.2±0.5%. The dried sprouts were pulverized at low temperature by a stone mill. The grain size was 35-170 meshes. This powdery sample of 10 kg was washed with 80% ethanol twice followed by filtering, 170 1 of water was added thereto, and the mixture was then heated to 85-90° C. to extract water. It was successively cooled to 40-50° C., and a small amount of protease was added thereto followed by stirring for 4 hours. The mixture was then heated to 80-90° C. to deactivate the protease simultaneously with water extraction. Thereafter, filtration was performed followed by spray draying to provide a rough powdery fucoidan-like polysaccharide composite.

Okinawa Tinocladia crassa-Derived Fraction

A rough powdery fucoidan-like polysaccharide composite obtained by performing the same raw material treatment as the fucoidan fraction extraction-fractionated from sprouts of wakame was used.

Agaricus (Agaricus Blajei Murrill)

The growing area of the spawn is Brazil. The one obtained by incubating the spawn in a bagasse culture medium, and then extracting the mycelium followed by powdering was used.

2. Preparation of Mixture

Mixed powders of the Okinawa Tinocladia crassa-derived fucoidan fraction and the Agaricus were variously blended to the fucoidan fraction extraction-fractionated from sprouts of wakame within the range of 1-90%, and a compounded mixture drinkable without unpleasant feeling in a sensitivity test was obtained. The compounded mixture was obtained by mixing the Agaricus powder to a mixture consisting of 15-35% of the fucoidan fraction extraction-fractionated from wakame sprouts and 85-65% of the fucoidan fraction extraction-fractionated from Okinawa Tinocladia crassa in a content of 10-20% based on the whole weight of the mixture.

3. Efficacy Test of Mixture

The mixture determined to be the easiest to drink in the sensitivity test contains a relatively small amount of the wakame sprout-derived fucoidan excellent in efficacy and a large quantity of the Okinawa Tinocladia crassa-derived fucoidan. Therefore, in order to manifest the function of the compounded mixture as bioprotective accelerator and immunostimulator and the influence by the additive function of the Agaricus, a feed supplemented with 2% of the compound of the present invention was given to experimental rats, and the anti-obesity effect, effect on blood properties, effect on serum cholesterol content and the like thereby were examined in a fraction section of Example. For comparison, the fraction section of a feed not supplemented with the compound of the present invention was taken as Comparative Example.

Table 1 shows the mixing ratios of the feeds used in this experiment.

TABLE 1

| | Comparative Example | Example |
|---|---|---|
| Component | (%) | (%) |
| α-Cornstarch | 52.18 | 50.8 |
| Casein | 25 | 24.9 |
| Cellulose | 7 | 6.6 |
| Corn oil | 6 | 6 |
| Sucrose | 5 | 5 |
| Mineral Mix* | 3.5 | 3.5 |
| Vitamin Mix* | 1 | 1 |
| Colin chloride | 0.2 | 0.2 |
| Compound of the Present Invention | 0 | 2 |
| Salt | 0.12 | 0 |
| Total | 100 | 100 |

(1) Anti-Obesity Effect

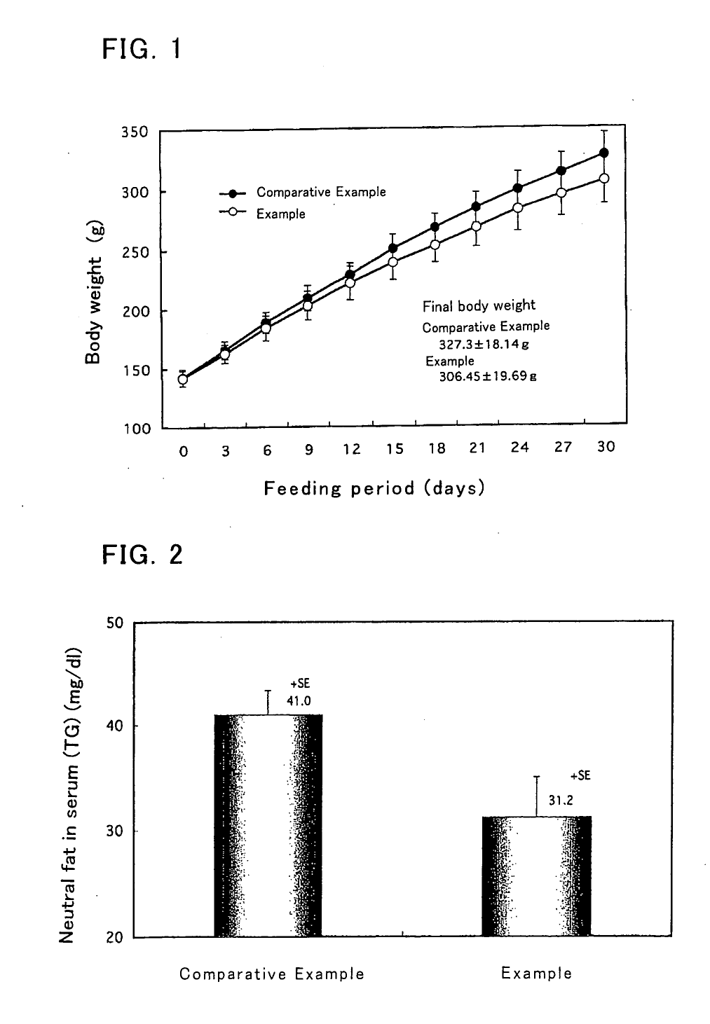

Rats (Wistar, male, 5-week-old) were fed on the experimental feeds for 30 days, and their effects on body weight change, blood properties and fat tissue weight were examined. The examination result is shown in FIG. 1. The bars in the graph show standard errors. As shown in the drawing, the weights of the rats fed on the compounded powder according to the fucoidan-based health food of the present invention fluctuated a little less, and the gain of body weight was also significantly reduced.

(2) Effect on Blood Properties

The blood was collected from the rats to obtain serums, and the neutral fat in serum was examined. The result is shown in FIG. 2. From the comparison between Example and Comparative Example in the drawing, it is found that the administration of the feed containing the fucoidan-based health food of the present invention is effective for the reduction in neutral fat (triglyceride) content.

(3) Effect on Serum Cholesterol Content

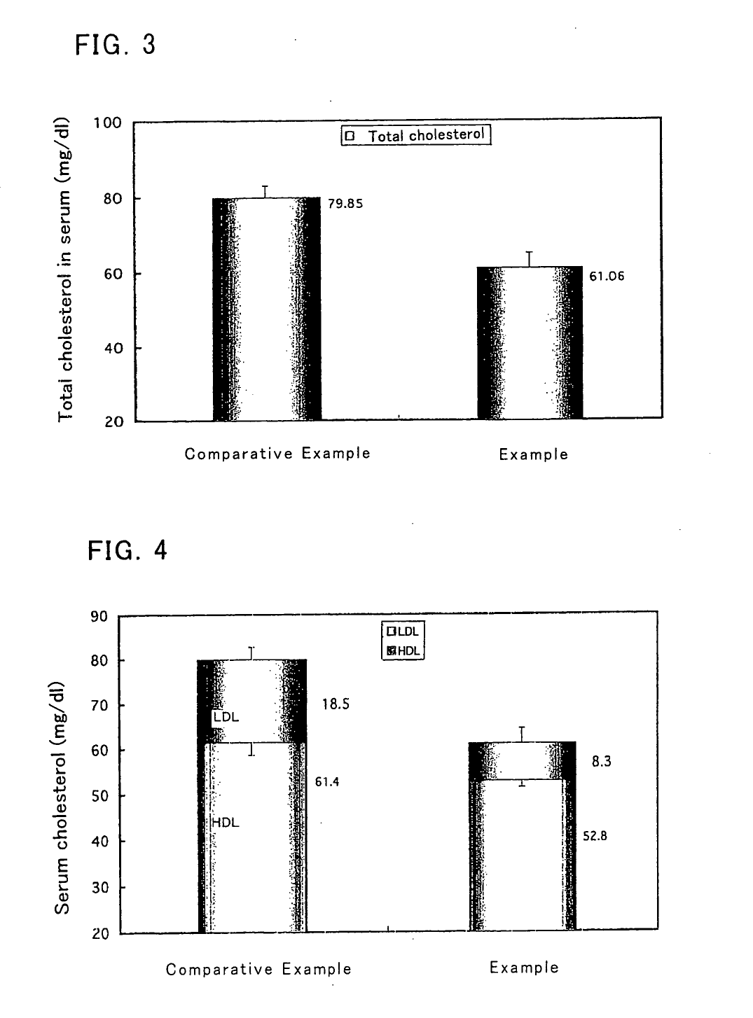

The influence on the cholesterol contents in the rat serums was examined. The result is shown in FIG. 3. As is apparent from the comparison between Example and Comparative Example in the drawing, the total cholesterol content was significantly reduced by the administration of the fucoidan-based health food of the present invention.

(4) Effect on HDL- and LDL-Cholesterol Contents

The HDL- and LDL-cholesterol contents in the rat serums were examined. The result is shown in FIG. 4. From the comparison between Example and Comparative Example in the drawing, the LDL-cholesterol content that is said to be physiologically bad and plays a role in obesity was significantly reduced by the administration of the fucoidan-based health food of the present invention without affecting the HDL-cholesterol content, and improvements in blood properties were observed. Thus, the fucoidan-based health food of the present invention was confirmed to be useful for maintenance of health.

(5) Effect on Fat Tissue Weight

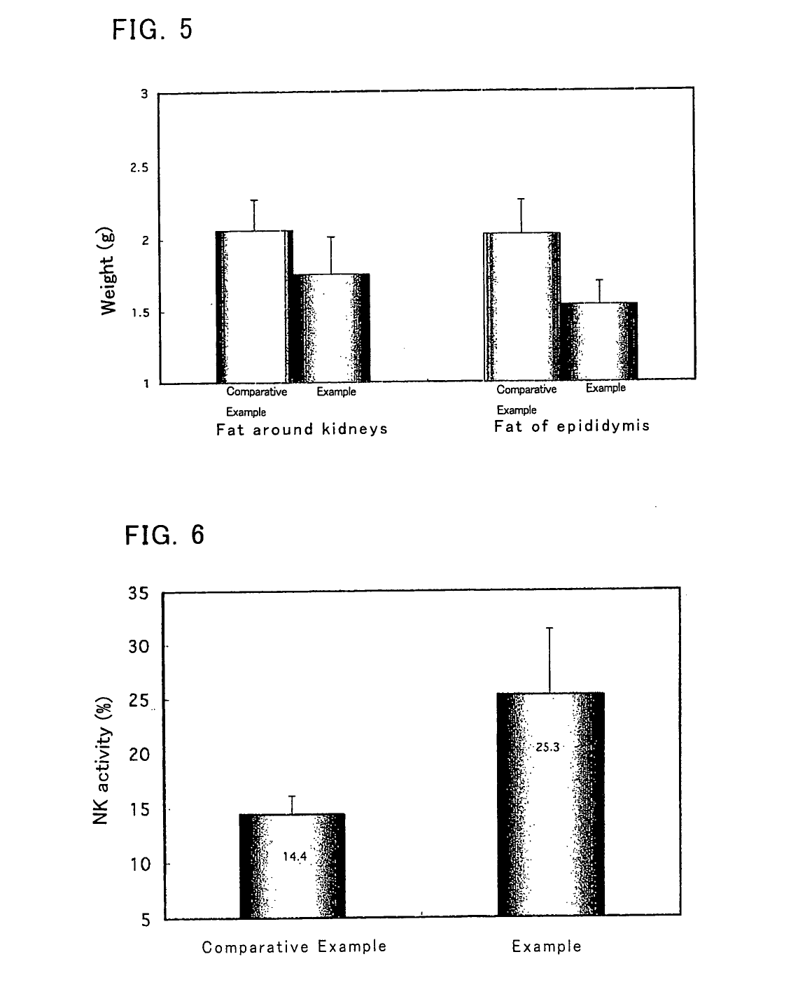

The fat tissue weights of the rats were examined in the same manner. The result is shown in FIG. 5. The fat tissues used were fat tissues around kidneys and fat tissues around epididymis. As shown in the drawing, the fat tissue weights of the both were reduced more in Example, which shows that the administration of the fucoidan-based health food of the present invention is effective for the reduction in fat tissue.

(6) Effect on Natural Killer Activity

It is conceivable that cancer might be prevented by enhancing the natural killer activity having the function of finding out and killing cancerous cells or enhancing the bioprotective ability. Therefore, the increase in NK activity by the fucoidan-based health food of the present invention was examined.

\mathring{1} Preparation of NK Cells

Preparation of Splenic Cells:

BALB/C mice (5-week-old, male) were fed on the feed supplemented with 2% of the fucoidan-based health food of the present invention shown in Table 1 for 20 days to obtain the spleen. A floating solution of cells was obtained from the spleen. A hypotonic buffer was added thereto to hemolyze and remove red blood cells, whereby splenic cells were obtained. These cells were put in a dish with RPMI culture medium, incubated at 37° C. with 5% carbon dioxide for 3 hours followed by removal of adhered cells to form splenic lymphocytes containing NK cells.

\mathring{2} Preparation of YAC-1 Cell (Lymphomatous Cell, Target Cell)

YAC-1 Cells (target cells) that are the target of the NK cells (effector cells) were separately incubated.

\mathring{3} Measurement of NKActivity

A cytotoxic ELISA assay system (Cytotox 96, produced by Promega) using Lactate dehydrogenase (LDH) that releases affected cells as a marker was used for the measurement of NK activity. The above operation was aseptically performed. The NK activity of the spleens of the mice that ate the fucoidan-based health food of the present invention for 20 days is shown in FIG. 6. As shown in the drawing, the activity in Example of the present invention tend to be high, compared with in Comparative Example, and the fucoidan-based health food of the present invention was confirmed to be effective for enhancing the NK activity, and enhanced in the bioprotective ability.

\mathring{4} Antitumor Activity

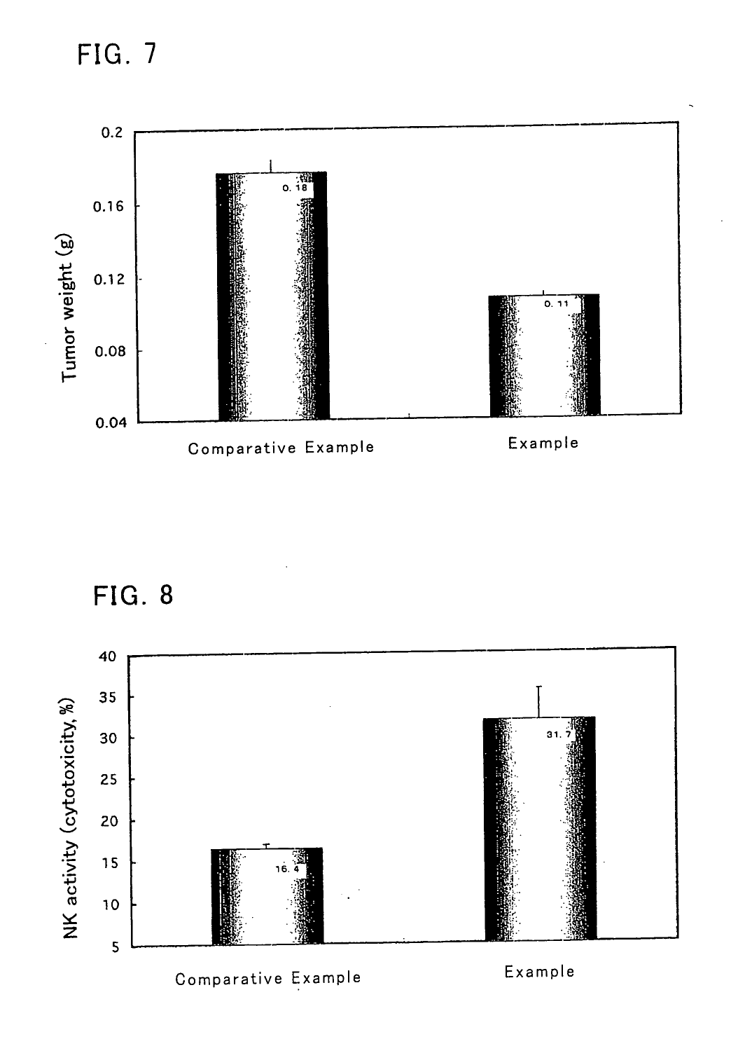

Sarcoma 180 tumor cells were subcutaneously administered to the kidneys of mice (BALB/C male, 5-week-old) (5×106 cells/mouse), and the mice were fed on the feed supplemented with the fucoidan-based health food of the present invention for 20 days. The mice were dissectioned, and the weight of the subcutaneous tumor of the kidneys was measured and compared with that of the control section. The result is shown in FIG. 7. As shown in the drawing, the reduction in tumor weight was apparently observed in the administered section, and the fucoidan-based health food of the present invention was thus confirmed to be effective for inhibition of tumor multiplication.

\mathring{5} Natural Killer (NK) Activity of Tumid Mouse

The NK activity in the splenic floating cells of the tumid mice was measured. The measurement result is shown in FIG. 8. The NK activity was confirmed to increase by the administration of the compound of the present invention even in the tumid mice with the tumor cells subcutaneously transplanted, and it could be concluded that this functions to inhibit the tumor multiplication.

\mathring{6} Inhibiting Effect of Hyperglycemia

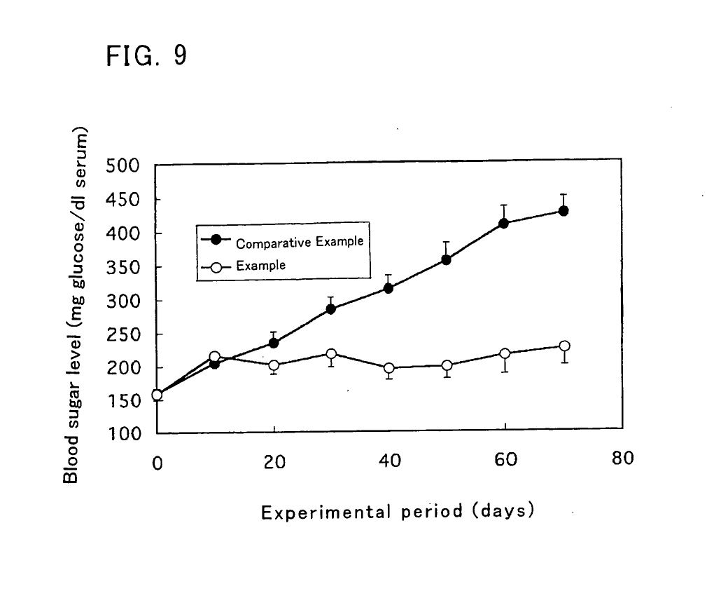

Model mice of insulin-independent diabetes, KK-Ay mice (5-week-old, male, Clea Japan) were fed on the feeds shown in Table 1 for 70 days. The blood was collected from the tails thereof every 10 days to measure the blood sugar levels, respectively. The measurement result is shown in FIG. 9. The measurement of blood sugar level was carried out by an enzymatic electrode method using Gluco Card (Arkary, GT-1640, Kyoto). Consequently, the blood sugar level of the mice of Comparative Example rises according to the feeding period, and reaches 400 mg glucose/dl serum after 70 days, while the mice of Example keep low blood sugar levels of 200 mg/dl that is about a half, which shows that the fucoidan-based health food of the present invention strongly inhibits hyperglycemia.

Effect of the Invention

The fucoidan-based health food of the present invention can be enhanced in the body weight control effect and the reducing effect of fat tissue weight, neutral fat (triglyceride) in serum, low-density lipoprotein, and total cholesterol content by being mixed with the fucoidan derived from Okinawa Tinocladia crassa, and further enhanced in the anticancer effect possessed also by Agaricus.

The fucoidan-based health food of the present invention can be provided to a human body regardless of the shape.

1. A fucoidan-based health food having the effect of inhibiting hyperglycemia and keeping blood sugar low, which is obtained by mixing an Agaricus powder to a mixture consisting of 1-90% of a fucoidan fraction extraction-fractionated from sprouts of wakame (Undaria pinnatifida) and the remainder of a fucoidan fraction extraction-fractionated from Okinawa Tinocladia crassa.

2. A fucoidan-based health food according to claim 1, wherein the Agaricus powder is mixed to a mixture consisting of 15-35% of the fucoidan fraction extraction-fractionated from sprouts of wakame and 85-65% of the fucoidan extraction-fractionated from Okinawa Tinocladia crassa in a content of 10-20% based on the whole amount of the mixture.
